(19) United States
(12) Patent Application Publication
(10) Pub. No.: US 2005/0048057 A1
(43) Pub. Date: Mar. 3, 2005

(54) Anti-human vitronectin antibody and methods for making the same

(75) Inventors: Duane E. Day, Novi, MI (US); Chad A. Link, Lansing, MI (US)

Correspondence Address: FITZPATRICK CELLA HARPER & SCINTO, 30 ROCKEFELLER PLAZA, NEW YORK, NY, 10112, US

(73) Assignee: Molecular Innovations

(21) Appl. No.: 10/886,204

(22) Filed: Jul. 7, 2004

Related U.S. Application Data
(60) Provisional application No. 60/487,041, filed on Jul. 11, 2003.

(51) Int. Cl. A61K 39/395; C12P 21/04; C12N 5/06

(52) U.S. Cl. 424/146.1; 530/388.26; 800/6; 435/70.21; 435/338

(57) ABSTRACT

The present invention relates to a method for raising a monoclonal antibody against human vitronectin and the purified anti-vitronectin antibody produced by this method. The antibody of the present invention is useful as both a research tool and as an agent for the diagnosis and treatment of cancers, cardiovascular diseases, particularly atherosclerosis and restenosis, osteoporosis, and inflammatory diseases.

### FIELD OF THE INVENTION

The present invention relates to hybridoma cell lines which produce a monoclonal antibody against human vitronectin and purified anti-vitronectin monoclonal antibody. The monoclonal antibody of the present invention is made by immunizing a mouse in which the endogenous mouse vitronectin gene has been inactivated through insertional mutagenesis. The purified anti-vitronectin antibody prepared by the method of the present invention is useful as a research tool in determining the pathophysiological bases of diseases and disorders associated with binding of vitronectin to its cellular receptor, and in diagnosis and treatment of such diseases and disorders, which include, but are not limited to, inflammation, cancer, cardiovascular disorders such as atherosclerosis and restenosis, and diseases wherein bone resorption is a causative or contributory factor, such as osteoporosis.

### BACKGROUND OF THE INVENTION

Vitronectin is a multifunctional glycoprotein present in blood and in the extracellular matrix. It binds glycosaminoglycans, collagen, plasminogen and the urokinase receptor, and also stabilizes the inhibitory conformation of plasminogen activation inhibitor-1. By its localization in the extracellular matrix and its binding to plasminogen activation inhibitor-1, vitronectin can potentially regulate the proteolytic degradation of this matrix. In addition, vitronectin binds to complement, heparin and thrombin-antithrombin III complexes, implicating its participation in the immune response and in the regulation of clot formation.

Vitronectin contains an RGD sequence, through which it binds to integrins, a superfamily of cell adhesion receptors. These transmembrane glycoprotein cell surface adhesion receptors include αᵥβ₃, the vitronectin receptor, and GPIIb/IIIa, the fibrinogen receptor. The vitronectin receptor is expressed on a number of cells, including endothelial, smooth muscle, osteoclast, and tumor cells, and, thus, it has a variety of functions. αᵥβ₃ expressed on the membrane of osteoclast cells mediates the bone resorption process and contributes to the development of osteoporosis. Ross et al., J. Biol. Chem., 1987:262:7703. αᵥβ₃ expressed on human aortic smooth muscle cells stimulates their migration into neointima, which leads to the formation of atherosclerosis and restenosis after angioplasty. Brown et al., Cardiovascular Res., 1994;28:1815. Additionally, a recent study has shown that an αᵥβ₃ antagonist is able to promote tumor regression by inducing apoptosis of angiogenic blood vessels. Brooks et al., Cell, 1994;79:1157. Thus, agents that would block the interaction of vitronectin with its receptor would be useful in treating diseases mediated by this interaction, such as osteoporosis, atherosclerosis, restenosis and cancer.

Because of the importance of angiogenesis to tumor growth and metastasis, a number of chemotherapeutic approaches are being developed to interfere with or prevent this process. One of these approaches involves the use of anti-angiogenic proteins such as angiostatin and endostatin. Angiostatin is a 38 kDa fragment of plasminogen that has been shown in animal models to be a potent inhibitor of endothelial cell proliferation. O'Reilly et al., Cell, 1994;79:315-328. Endostatin is a 20 kDa C-terminal fragment of collagen XVIII that has also been shown to be a potent inhibitor. O'Reilly et al., Cell, 1997;88:277-285. Systemic therapy with endostatin has been shown to result in strong anti-tumor activity in animal models. However, human clinical trials of these two chemotherapeutic agents of biological origin have been hampered by lack of availability.

Another approach to anti-angiogenic therapy is to block the binding of vitronectin or fibronectin to the αᵥβ₃ or GPIIb/IIIa integrins, respectively. Alig et al., EP 0 381 033; Hartman et al., EP 0 540,334; Blackburn et al., WO 93/08174; Bondinell et al., WO 93/00095; Blackburn et al., WO 95/104057; Egbertson et al., EP 0 478 328; Sugihara et al., EP 529,858; Porter et al., EP 0 542 363; Fisher et al., EP 0 635 492 disclose certain compounds that are useful for inhibiting the fibrinogen receptor. PCT/US95/08306, filed Jun. 29, 1995 (SmithKline Beecham Corp.) and PCT/US95/08146, filed Jun. 29, 1995 (SmithKline Beecham Corp.) disclose vitronectin receptor selective antagonists. However, there are few reports of compounds which are potent vitronectin receptor antagonists. Thus, it is desirable to provide anti-angiogenic pharmaceuticals that exhibit specificity toward the vitronectin/αᵥβ₃ interaction.

In accordance with the present invention, it has been discovered that 1) a purified monoclonal antibody can be produced through the immunization with purified human vitronectin of transgenic mice in which the mouse vitronectin gene has been inactivated through insertional mutagenesis; 2) splenocytes from the immunized mice may be isolated and employed for the production of monoclonal antibodies specific for human vitronectin; and 3) the purified anti-human vitronectin antibodies block the binding of human vitronectin to αᵥβ₃ on the surface of human smooth muscle cells or human endothelial cells. Accordingly, a reagent has been produced that is useful in elucidating the pathogenesis of various diseases related to the binding of vitronectin to αᵥβ₃, such as cancer, cardiovascular diseases, osteoporosis and inflammatory diseases, in identifying endogenous or environmental factors that could contribute to the etiologies of these diseases, and in developing effective therapies for the prevention and/or treatment of these diseases.

### SUMMARY OF THE INVENTION

The present invention is based, at least in part, on the observation that the purified anti-human vitronectin antibodies block the binding of human vitronectin to αᵥβ₃ on the surface of human smooth muscle cells or human endothelial cells. Thus, the present invention relates to the purified anti-vitronectin antibody produced by this method. The present invention further relates to a method for raising a monoclonal antibody against human vitronectin by immunization of a mouse in which the endogenous mouse gene encoding vitronectin has been inactivated through insertional mutagenesis. The purified anti-vitronectin antibody prepared by the method of the present invention is useful as a research tool in determining the pathophysiological bases of diseases and disorders associated with binding of vitronectin to its cellular receptor, and in the diagnosis and treatment of such diseases and disorders which include but are not limited to, inflammation, cancer, cardiovascular disorders such as atherosclerosis and restenosis, and diseases wherein bone resorption is a factor, such as osteoporosis.

### DESCRIPTION OF THE DRAWINGS

FIG. 1. Effects of eight candidate anti-human vitronectin antibodies on the adhesion of smooth muscle cells to immobilized human vitronectin.

FIG. 2. Amino acid sequence of human vitronectin protein.

### DETAILED DESCRIPTION OF THE INVENTION

The present invention relates to hybridoma cell lines which produce monoclonal antibodies against human vitronectin and to purified anti-human vitronectin antibody. The present invention further relates to a method for raising a monoclonal antibody against human vitronectin by immunization of a mouse in which the endogenous mouse gene encoding vitronectin has been inactivated through insertional mutagenesis (hereinafter “vitronectin KO mice”). The antibody can be used to inhibit vitronectin binding to the αᵥβ₃ integrin and blocking the physiologic responses of receptor binding. The monoclonal antibody of the present invention therefore may be used as a diagnostic or therapeutic agent for medical conditions in which vitronectin has been implicated.

In accordance with the method of the present invention, vitronectin KO mice are immunized with human vitronectin, and antibody-producing B-lymphocytes are harvested from their spleens. Preferably the immunization is conducted periodically, and in various amounts to induce in vivo generation of anti-human vitronectin activity. Purified or non-purified human vitronectin may be used, either alone or mixed with complete or incomplete Freund's adjuvant. Immunization may occur through a single intramuscular or intraperitoneal injection, or through a combination of injections by different routes and/or at different sites, either simultaneously or serially, in the hind legs and in the peritoneal cavity.

In preferred embodiments, vitronectin KO mice are immunized with human vitronectin by intraperitoneal (i.p.) injection at doses from 10-50 micrograms of purified human vitronectin per animal. The primary immunization is carried out in the presence of complete Freund's adjuvant and boosts are provided in incomplete Freund's at two and 6 weeks after the initial immunization. A final boost in incomplete Freund's is administered i.p. four days prior to the harvesting of spleen cells for cell fusion and hybridoma development.

Spleen cells from the vitronectin KO mice immunized with human vitronectin are then removed and single cell suspensions are prepared therefrom. The splenocytes are cultured in a tissue culture medium supplemented with various additives. A B cell mitogen is then added to the culture medium to activate the “immune” spleen cells.

Once activated, the murine B lymphocytes prepared as described above are fused with malignant, murine myeloma cells by mixing the two different types of cells together and then pelleting the mixture. The cell pellet is resuspended in a tissue culture medium which also contains a fusing agent.

In preferred embodiments, the myeloma cells to which the splenocytes are fused are murine myeloma P3X63-Ag8.653 cells. Artisans of ordinary skill would recognize that other cell lines also may be suitable alternatives to the P3X63-Ag8.653 cell line.

Fusing agents may include various types of condensation polymers of ethylene oxide and water, such as polyethylene glycol (hereinafter “PEG”) 1500. Other fusing agents include deoxyribonucleic acid (hereinafter “DNA”) transforming viruses, such as Sendai virus or the fusion protein obtained therefrom. For optimum fusion, the quantity and concentration of the fusing agent must be controlled. For instance, if PEG 1500 is used to fuse immunized BALB/c mouse splenocytes with P3X63-Ag8.653 myeloma cells, this fusing agent should comprise about 40% (weight/volume). However, the volume of PEG 1500 may range from 0.5 to 3 milliliters and the concentration of PEG 1500 may vary from 35% to 60% weight/volume of culture medium. In a preferred embodiment, the splenocytes from the immunized vitronectin KO mice are resuspended in a fusing agent consisting of 42% polyethylene glycol (PEG 4000) and 15% dimethylsulfoxide (DMSO) in phosphate buffered saline. Again, artisans of ordinary skill would recognize that these parameters may be varied according to the fusing agent employed and the cell types to be fused.

Thereafter, the cell solution is pelleted again and resuspended in another protein-containing medium which is supplemented with various additives, feeder cells and selective suppressing agents that preclude the growth of unfused myeloma cells, thereby liberating an anti-human vitronectin antibody producing hybrid cell. Alternatively, anti-human vitronectin antibody may be expanded by injecting the hybridoma cells from the cell pellet into the peritoneal cavities of mice and thereafter collecting the interperitoneal ascites which contain high concentrations of anti-human vitronectin antibody. Artisans of ordinary skill will recognize that many variations of these procedures may be employed to obtain the anti-human vitronectin-producing cell lines of the present invention.

The present invention also concerns identifying potent anti-human vitronectin antibody-producing cell lines by cloning hybrid cell lines found to produce this antibody constitutively. Cloning is accomplished by a limiting dilution procedure wherein anti-human vitronectin antibody-producing hybrid cells are individually cultured in vitro in culture medium containing feeder cells and selective suppressing agents which prevent the growth of unfused myeloma cells.

In the process of the present invention, vitronectin KO mice have been utilized as a source of anti-human vitronectin antibody-producing B lymphocytes. Activated spleen cells from these human vitronectin-immunized mice have been fused with the P3X63-Ag8.653 murine myeloma cell line to produce several hybrid cell lines capable of constitutive anti-human vitronectin antibody production. Other myeloma cell lines capable of being used in the present invention include SP2, NS-1, other P3 variants, XC3, Ag8 and other drug-marked BALB/c mouse myeloma cell lines.

Cloning of the cells obtained through fusion of activated spleen cells with murine myeloma cells has resulted in the identification of several clonal lines which are capable of producing anti-human vitronectin antibody, including the cell line labeled as 615.1D1. Subcloning the 615.1D1 clonal cell line has isolated an even more potent antibody source, designated as 615.1D1.44. The present invention thus provides for the 615.1D1 and 615.1D1.44 hybridoma cell lines capable of producing anti-human vitronectin antibodies. The hybridoma cell line 615.1D1.44 of the present invention was deposited with the American type Culture Collection (ATCC®) on Jul. 8, 2003 and has been given ATCC deposit number ______.

The present invention further provides for the anti-human vitronectin monoclonal antibodies produced by the clonal 615.1D1 and 615.1D1.44 hybridoma cell lines. Specifically, anti-human vitronectin antibody from hybridoma clone 615.1D1.44 is produced by injecting the hybridoma cells from the cell pellet into the peritoneal cavities of BALB/c mice. The mice are “primed” by intraperitoneal injection of 0.5 milliliters of 2,6,10,14-tetramethyl-pentadecane (pristane) and subsequently immunosuppressed by intramuscular injection of hydrocortisone semi-succinate (3 mg/mouse) fourteen days (day 14) post-pristane injection and gamma irradiation (600 rads) on day 15, followed by injection of hybridoma cells on day 17. Intraperitoneal ascites containing high concentrations of anti-human vitronectin develops and is collected.

Within the context of the present invention, antibodies are understood to be monoclonal antibodies. However, the present invention further provides for polyclonal antibodies, antibody fragments (e.g. Fab, and F(ab′)₂) and recombinantly-produced binding partners which specifically bind to human vitronectin. Polyclonal antibodies against selected antigens may be readily generated by one of ordinary skill in the art from a variety of warm-blooded animals such as horses, cows, various fowl, rabbits or rats.

The specificity of the anti-human vitronectin antibody produced by the parent and clonal cell lines may be ascertained by testing the capacity of the antibody to bind to immobilized vitronectin. In preferred embodiments, specificity of candidate antibodies is determined by indirect ELISA (enzyme-linked immunosorption assay) using immobilized purified human vitronectin under standard conditions.

The biological efficacy of the anti-human vitronectin antibody produced by the parent and clonal cell lines may be established by determining the ability of the antibodies to inhibit the ability of vitronectin to bind to the αᵥβ₃ integrin. In a preferred embodiment, the biological efficacy of the anti-human vitronectin antibody may be determined by measuring the ability of the antibody to inhibit binding of smooth muscle cells to immobilized human vitronectin.

The screening procedure outlined above identified the hybridoma cell line capable of producing mAb 615-1D1. This anti-human vitronectin monoclonal antibody may be employed in a competitive binding assay to identify additional antibodies or small molecules that bind to vitronectin and inhibit the effects of the binding of vitronectin to αᵥβ₃. Thus, the present invention provides for an assay for identifying inhibitors of vitronectin activity.

As discussed briefly above, binding of vitronectin to αᵥβ₃ stimulates migration of vascular smooth muscle, proliferation of vascular endothelial cells, induces angiogenesis, and blocks bone resorption. Thus, the anti-human vitronectin antibody of the instant invention is useful for inhibiting these processes. Specifically, this antibody is useful in the diagnosis and treatment of angiogenic diseases, inflammatory diseases such as macular degeneration, age-related macular degeneration, arthritis, rheumatoid arthritis, atherosclerosis, diabetic retinopathy, thyroid hyperplasia, Grave's disease, hemangioma, neovascular glaucoma, pyrogenic granuloma, scleroderma, synovitis, trachoma or psoriasis, proliferative disorders such as cancers, arteriovenous malformations (AVM), meningioma, vascular restenosis, angiofibroma, dermatitis, endometriosis, hypertrophic scars, or vascular adhesions.

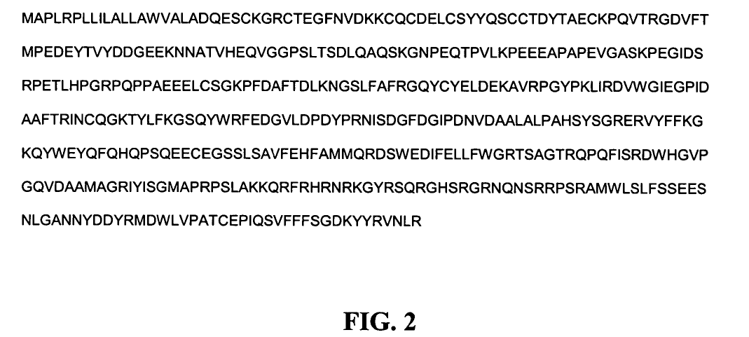

In one embodiment, the antibody is prepared against the human vitronectin protein having an amino acid sequence of SEQ ID NO:1, (see FIG. 2) or an antigenic fragment thereof. The antibodies of the present invention can be either monoclonal antibodies or polyclonal antibodies. In one embodiment, the antibody is a monoclonal antibody that is a chimeric antibody.

An immortal cell line that produces a monoclonal antibody of the present invention is also part of the present invention. In a specific embodiment of this immortal cell line, the monoclonal antibody is prepared against the human vitronectin protein having an amino acid sequence of SEQ ID NO:1 or an antigenic fragment thereof.

Methods of purifying the anti-human vitronectin antibody are also part of the present invention, as are the purified antibodies themselves.

The purified antibodies may be incorporated into various pharmaceutical compositions, and these compositions may be administered by any means known in the art to achieve the intended purpose. Amounts and regimens for the administration of these compositions can be determined readily by those with ordinary skill in the clinical art of treating any of the particular diseases.

Administration may be by parenteral, subcutaneous (s.c.), intravenous (i.v.), intramuscular (i.m.), intraperitoneal (i.p.), transdermal, topical or inhalation routes. Alternatively, or concurrently, administration may be by the oral route. The dosage administered will be dependent upon the age, health, and weight of the recipient, kind of concurrent treatment, if any, frequency of treatment, and the nature of the effect desired.

Compositions within the scope of this invention include all compositions wherein the anti-human vitronectin antibody of the present invention is contained in an amount effective to achieve its intended purpose. While individual needs vary, determination of optimal ranges of effective amounts of each component is within the skill of the art. Typical dosages comprise 0.1 to 100 mg/kg/body weight, although other dosages outside of this range may be desirable for certain particular uses.

In addition to the biologically-active antibody, the new pharmaceutical preparations may contain suitable pharmaceutically-acceptable carriers comprising excipients and auxiliaries which facilitate processing of the active compounds into preparations which can be used pharmaceutically as is well known in the art. Suitable solutions for administration by injection or orally, may contain from about 0.01 to 99 percent, active compound(s) together with the excipient.

Included in the scope of this invention are salts of the anti-human vitronectin antibody of the present invention. The term “salts” refers to both salts of carboxyl groups and to acid addition salts of amino groups of the protein or peptide. Salts of a carboxyl group include inorganic salts, for example, sodium, calcium, ammonium, ferric or zinc salts, and the like, and salts with organic bases such as those formed with amines, such as triethanolamine, arginine, or lysine, piperidine, procaine, and the like. Acid addition salts include salts with mineral acids such as hydrochloric or sulfuric acid, and salts with organic acids such as acetic or oxalic acid.

The pharmaceutical preparations of the present invention are manufactured in a manner which is itself known, for example, by means of conventional mixing, granulating, dissolving, or lyophilizing processes. Suitable excipients may include fillers, binders, disintegrating agents, auxiliaries and stabilizers, all of which are known in the art. Suitable formulations for parenteral administration include aqueous solutions of the proteins in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions that may contain substances which increase the viscosity of the suspension may also be employed.

The pharmaceutical formulation for systemic administration according to the invention may be formulated for enteral, parenteral or topical administration, and all three types of formulation may be used simultaneously to achieve systemic administration of the active ingredient.

As described for lung instillation, aerosolized solutions are used. In a sprayable aerosol preparations, the active purified monoclonal antibody of the invention may be administered in combination with a solid or liquid inert carrier material. This may also be packaged in a squeeze bottle or in admixture with a pressurized volatile, normally gaseous propellant. The aerosol preparations can contain solvents, buffers, surfactants, and antioxidants in addition to the protein of the invention.

For topical application, the monoclonal antibody of the present invention may be incorporated into topically-applied vehicles such as salves or ointments, which have both a soothing effect on the skin as well as a means for administering the active ingredient directly to the affected area.

The carrier for the active ingredient may be either in sprayable or nonsprayable form. Non-sprayable forms can be semi-solid or solid forms comprising a carrier indigenous to topical application and having a dynamic viscosity preferably greater than that of water. Suitable formulations include, but are not limited to, solutions, suspensions, emulsions, creams, ointments, powders, liniments, salves, and the like. If desired, these may be sterilized or mixed with auxiliary agents, e.g., preservatives, stabilizers, wetting agents, buffers, or salts for influencing osmotic pressure and the like. Examples of preferred vehicles for non-sprayable topical preparations include ointment bases, e.g., polyethylene glycol-1000 (PEG-1000); conventional creams such as HEB cream; gels; as well as petroleum jelly and the like. One particularly preferred cream is described below.

Other pharmaceutically acceptable carriers for the anti-vitronectin antibody made in accordance with the present invention are liposomes, pharmaceutical compositions in which the anti-vitronectin antibody is contained either dispersed or variously present in corpuscles consisting of aqueous concentric layers adherent to lipidic layers. The anti-vitronectin antibody is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature.

As will be appreciated by the skilled artisan, the anti-vitronectin antibody of the present invention may be useful for treating a variety of diseases and disorders associated with the binding of vitronectin to αᵥβ₃. including atherosclerosis, restenosis, to inhibit both local and metastatic tumor growth by inhibiting angiogenesis, and thrombosis.

The following nonlimiting examples serve to further illustrate the present invention.

### EXAMPLE

### Experimental Procedures

Purification of human vitronectin. Human native vitronectin was purified from fresh human plasma as described by Tornasini and Mosher (Tornasini and Mosher, Prog Haemostasis Thromb 1991;10:269-304), the entire disclosure of which is incorporated herein by reference.

Generation of vitronectin KO mice. The vitronectin KO mice were generated using standard gene targeting methods, as described in Zheng et al. Zheng X, Saunders T L, Camper S A, Samuelson L C, Ginsburg D. Proc Natl Acad Sci USA 1995;92:12426-12430. Briefly, the mouse vitronectin gene was isolated from a genomic library and cloned into a plasmid vector. A portion of the vitronectin gene was deleted. This construct was then used to transform mouse embryonic stem (ES) cells. Clones in which the intended homologous recombination event occurred were identified by Southern blotting. Selected clones were injected into mouse blastocysts, which were implanted into pseudopregnant female mice. Progeny mice demonstrating a relatively high number of mutant cells (i.e. those carrying the null allele) were identified by their coat color. Appropriate mice were mated, and the offspring of these mice were screened to determine if they had inherited the vitronectin null allele. Mice carrying the null allele were mated, and mice inheriting both null alleles (homozygous knockouts) were identified by standard genotyping methods.

Immunization of vitronectin KO mice with human vitronectin. Vitronectin KO mice were immunized with human vitronectin by intraperitoneal (i.p.) injection at doses from 10-50 micrograms antigen per animal. A primary immunization was carried out in the presence of complete Freund's adjuvant and boosts were provided in incomplete Freund's at intervals (week 2 and at week 6). A final boost in incomplete Freund's was administered i.p. four days prior to the harvesting of spleen cells for cell fusion and hybridoma development.

Generation of anti-human vitronectin producing hybridoma cells. Spleen cells from the vitronectin KO mice immunized with human vitronectin were removed and single cell suspensions were prepared. The isolated splenocytes were fused with the malignant, murine myeloma cell line P3X63-Ag8.653 by mixing the two different types of cells together at a ratio between 3-5 spleen cells per myeloma cell and pelleting the mixture. The cell pellet then was resuspended in a fusing agent consisting of 42% polyethylene glycol (PEG 4000) and 15% dimethylsulfoxide (DMSO) in phosphate buffered saline. The fused cell solution was pelleted again and resuspended in tissue culture medium IMDM (Iscove's modified DMEM) supplemented with 20% fetal bovine serum, L-glutamine, penicillin/streptomycin and feeder cells from the peritoneal lavage of one BALB/c mouse. The hybridoma selection agent HAT (100 mM hypoxanthine, 0.4 mM aminopterin, 16 mM thymidine) was added to the culture medium to preclude the growth of unfused myeloma cells.

The resulting cell suspension was plated in 96-well culture dishes. Cultures were maintained at 37° C., 7% CO₂ and fed with the HAT selection media on days 7, 9 and 12. The culture supernatants were screened for vitronectin antibody production at 14-18 days post-fusion as described below. Anti-human vitronectin antibody-producing hybrids were subcloned in 96-well culture dishes by limiting dilution at 0.5-2.5 cells/well in the presence of murine thymocyte feeder layers. High-titer subclones were expanded and cryopreserved, and may be used for in vivo antibody production in ascites fluid in mice.

Screening of the monoclonal antibodies produced by the hybridoma cell lines. The hybridoma cell lines generated as described above were screened for the production of monoclonal antibodies specific for human vitronectin. This screening procedure was performed using standard indirect ELISA (enzyme-linked immunosorbent assay) screening methods, in which purified human vitronectin was immobilized on the ELISA plate. See generally Current Protocols in Immunology, John Wiley & Sons, 1991 with annual updating, pp. 2.1.2-2.1.6.

Purification of the monoclonal antibodies. The antibodies produced by the hybridoma cell lines were of class immunoglobulin G (IgG). Thus, the antibodies could be quickly and easily purified by Protein A-affinity chromatography using standard methods. See e.g. Current Protocols in Immunology, John Wiley & Sons, 1991 with annual updating, pp. 2.7.4-2.7.6.

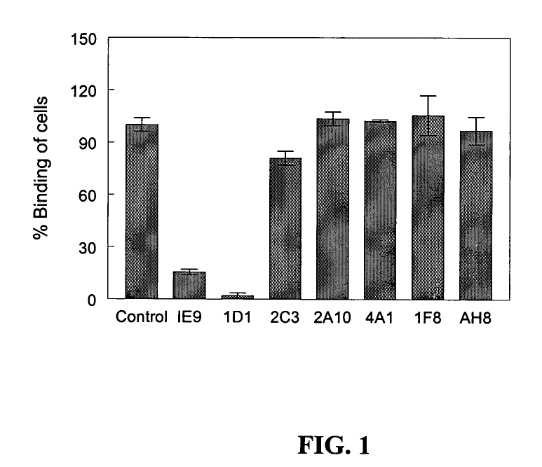

Testing of anti-human vitronectin monoclonal antibodies for biological efficacy. As shown in FIG. 1, eight candidate monoclonal antibodies raised against purified human vitronectin in vitronectin KO mice were screened for their ability to inhibit the adhesion of smooth muscle cells (SMC's) on immobilized human vitronectin. In these experiments, 24-well plates were coated with 1 microgram/ml of purified vitronectin for 1 hour at 37° C. followed by blocking with 1% BSA in TBS for 1 hour at 37° C. Ascites, (5 microliters in 0.4 ml 1% BSA in TBS) was then added to the wells and incubated for 1 hour at 37° C., followed by washing with TBS to remove any unbound antibodies. SMC's (ten thousand cells/ml, in DMEM and 1% BSA) were added to the wells and allowed to adhere for 45-60 min at 37° C. in and 10% CO2, followed by washing the unbound SMC's using TBS. Adherent cells were quantified by measuring acid phosphatase activity in the wells as described by Yang et al. Yang T T, Sinai P, Kain S R. Anal Biochem. 1996;241:103-8. An acid phosphatase assay for quantifying the growth of adherent and nonadherent cells). Briefly, cells were incubated with 0.1 M NaAcetate pH 5.5, containing 0.1% Triton-X-100 and 1 mg/ml paranitrophenyl-phosphate. After 1-2 hours incubation at 37° C., a sample was taken from the wells and neutralized using 1M Tris pH 8.8 and color was measured at 405 nm. The data shown in FIG. 1 are plotted as percentage of cell binding relative to the control sample, which contained no antibody. The results indicate that monoclonal antibody 615.1D1, produced by the 615.1D1 and 615.1D1.44 hybridoma cell lines, produces a nearly complete block of SMC binding to vitronectin.

Although the present invention has been described with reference to certain preferred embodiments, various modifications, alterations, and substitutions will be apparent to those skilled in the art without departing from the spirit and scope of the invention, as defined by the appended claims. All references cited herein are incorporated herein in their entireties.

1. An antibody which specifically binds to a mammalian protein having an amino acid sequence of SEQ ID NO: 1.

2. The antibody according to claim 1, wherein the antibody is selected from the group consisting of an isolated polyclonal antiserum, a preparation of purified polyclonal antibodies, and a preparation containing one or more monoclonal antibodies.

3. A hybridoma cell line 615.1D1.

4. A hybridoma cell line 615.1D1.44.

5. A monoclonal antibody produced by the hybridoma cell line of claim 3 or 4.

6. An antigen binding fragment of the monoclonal antibody of claim 5.

7. The monoclonal antibody according to claim 5, coupled to a detectable label or a substance having toxic or therapeutic activity.

8. The antigen binding fragment according to claim 6, coupled to a detectable label or a substance having toxic or therapeutic activity.

9. A method of making a hybridoma cell line producing a monoclonal antibody against human Vitronectin comprising:
(a) providing a vitronectin knockout mouse;
(b) injecting the mouse with human vitronectin; and
(c) obtaining hybridoma cells from the mouse wherein the hybridoma cells produce a monoclonal antibody against human vitronectin.

10. A hybridoma cell line made by the process comprising:
(a) providing a vitronectin knockout mouse;
(b) injecting the mouse with human vitronectin; and
(c) obtaining hybridoma cells from the mouse, wherein the hybridoma cells produce a monoclonal antibody against human vitronectin.

11. A method of making purified monoclonal antibody against human Vitronectin comprising:
(a) providing a vitronectin knockout mouse;
(b) injecting the mouse with human vitronectin;
(c) obtaining hybridoma cells from the mouse, wherein the hybridoma cells produce a monoclonal antibody against human vitronectin; and
(d) purifying the monoclonal antibody from the hybridoma cells.

12. A purified monoclonal antibody against human vitronectin made by the process comprising:
(a) providing a vitronectin knockout mouse;
(b) injecting the mouse with human vitronectin;
(c) obtaining hybridoma cells from the mouse, wherein the hybridoma cells produce a monoclonal antibody against human vitronectin; and
(d) purifying the monoclonal antibody from the hybridoma cells.

13. A method of inhibiting human vitronectin binding to the human vitronectin receptor αᵥβ₃, said method comprising contacting a human cell population including cells that express αᵥβ₃ with a composition comprising a biologically effective amount of at least a first anti-human vitronectin antibody or an antigen-binding fragment of said antibody.

14. A method of inhibiting human vitronectin-induced proliferation of human endothelial cells comprising contacting a biological tissue comprising a population of human endothelial cells with a composition comprising a biologically effective amount of at least a first anti-human vitronectin antibody or an antigen-binding fragment of said antibody.

15. A method of inhibiting human vitronectin-induced proliferation of human smooth muscle cells comprising contacting a biological tissue comprising a population of human smooth muscle cells with a composition comprising a biologically effective amount of at least a first anti-human vitronectin antibody or an antigen-binding fragment of said antibody.

16. A method of inhibiting angiogenesis in a human subject comprising contacting a population of potentially angiogenic blood vessels with at least a first anti-angiogenic composition comprising a biologically effective amount of at least a first anti-human vitronectin antibody or an antigen-binding fragment of said antibody.

17. A method of inhibiting restenosis in a human subject comprising contacting a population of potentially restenotic blood vessels with at least a first anti-restenotic composition comprising a biologically effective amount of at least a first anti-human vitronectin antibody or an antigen-binding fragment of said antibody.

18. A method of inhibiting inflammation in a human subject comprising contacting a population of potentially inflammatory cells with at least a first anti-inflammatory composition comprising a biologically effective amount of at least a first anti-human vitronectin antibody or an antigen-binding fragment of said antibody.

19. A method of inhibiting osteoporosis in a human subject comprising contacting a population of potentially pro-osteoporotic osteoclasts with at least a first anti-osteoporotic composition comprising a biologically effective amount of at least a first anti-human vitronectin antibody or an antigen-binding fragment of said antibody.

20. A method of inhibiting angiogenesis-dependent cancer in a human subject comprising contacting a population of potentially angiogenesis-dependent cancer cells with at least a first anti-angiogenic composition comprising a biologically effective amount of at least a first anti-human vitronectin antibody or an antigen-binding fragment of said antibody.

21. A method for treating a human subject that has, or is at risk for developing, a vascularized solid tumor, a metastatic tumor or metastases from a primary tumor, comprising administering to said human at least a first pharmaceutical composition that comprises at least a first purified, unconjugated anti-human vitronectin antibody, or an antigen-binding fragment thereof, that significantly inhibits vitronectin binding to the αᵥβ₃ integrin cell adhesion molecule, thereby inhibiting angiogenesis within said vascularized solid tumor, said metastatic tumor or said metastases from a primary tumor.

22. The method of claims 13, 14, 15, 16, 17, 18, 19, 20 or 21, wherein said at least a first anti-human vitronectin antibody is a monoclonal antibody or an antigen-binding fragment thereof.

23. The method of claim 22, wherein said at least a first anti-human anti-vitronectin antibody is an IgG antibody or an IgM antibody.

24. The method of claims 13, 14, 15, 16, 17, 18, 19, 20 or 21, wherein said at least a first anti-human vitronectin antibody is an scFv, Fv, Fab′, Fab, diabody, linear antibody or F(ab′)₂ antigen-binding fragment of an antibody.

25. The method of claim 24 wherein said at least a first anti-human vitronectin antibody is a dimer, trimer or multimer of said antibody or antigen-binding fragment thereof.

26. The method of claim 24 wherein said at least a first anti-human vitronectin antibody is a human, humanized or part-human antibody or antigen-binding fragment thereof.

27. The method of claim 24 wherein said at least a first anti-human vitronectin antibody is a chimeric antibody.

28. The method of claim 24 wherein said at least a first anti-human vitronectin antibody is a recombinant antibody.

29. The method of claims 13, 14, 15, 16, 17, 18, 19, 20 or 21, wherein said at least first anti-human vitronectin antibody specifically binds to a mammalian protein having an amino acid sequence of SEQ ID NO:1.

30. The method of claim 29 wherein the antibody is selected from the group consisting of an isolated polyclonal antiserum, a preparation of purified polyclonal antibodies, and a preparation containing one or more monoclonal antibodies.

31. The method of claims 13, 14, 15, 16, 17, 18, 19, 20 or 21, wherein said at least first anti-human vitronectin antibody is an antibody produced by hybridoma cell line 615.1D1.

32. The method of claims 13, 14, 15, 16, 17, 18, 19, 20 or 21, wherein said at least first anti-human vitronectin antibody is an antibody produced by hybridoma cell line 615.1D1.44.

33. The method of claims 13, 14, 15, 16, 17, 18, 19, 20 or 21, wherein said at least first anti-human vitronectin antibody is made by the process comprising:
(a) providing a vitronectin knockout mouse;
(b) injecting the mouse with human vitronectin;
(c) obtaining hybridoma cells from the mouse, wherein the hybridoma cells produce a monoclonal antibody against human vitronectin; and
(d) purifying the monoclonal antibody from the hybridoma cells.


SEQUENCE LISTING (1 sequence)

SEQ ID NO: 1 - protein, 450 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRAMWLSLFSSEESNLGANNYDDYRMDWLVPATCEPIQSVFFFSGDKYYRVNLR